(19) United States
(12) Patent Application Publication
(10) Pub. No.: US 2005/0047960 A1
(43) Pub. Date: Mar. 3, 2005

(54) Novel method for treatment of mold contamination

(76) Inventor: Michael R. Cushman, Punta Gorda, FL (US)

Correspondence Address: Catherine J. Walsh, 1875 Riviera Circle, Sarasota, FL, 34232, US

(21) Appl. No.: 10/925,836

(22) Filed: Aug. 25, 2004

Related U.S. Application Data
(60) Provisional application No. 60/498,211, filed on Aug. 26, 2003.

(51) Int. Cl. A61L 2/22

(52) U.S. Cl. 422/32; 422/27

(57) ABSTRACT

The present invention describes a novel method for treating mold contamination in homes or other buildings. This invention relates to a method using ozone in the decontamination and/or remediation of mold contamination in homes or other buildings. The method includes generating an ultrafine, stable, mist or fog comprised of very small water droplets containing dissolved ozone directly onto building surfaces or into building areas contaminated with mold. The ultrafine mist or fog can be generated using a commercially available nebulizer or generator to disperse the uniform, ultrafine mist or fog. The dispersal of ozone dissolved in very small water droplets utilized in this method is effective in destroying mold growth, is effective in very small areas, such as cracks and crevices and other small openings, such as inside walls and duct work and the like, but does not damage building structures, and is not harmful to plants or animals. The present invention is an effective, economical, and environmentally friendly method for the treatment of mold contamination in homes and other buildings.

### CROSS-REFERENCE TO RELATED APPLICATIONS

This application is an application under 35 USC 111(a) and claims priority under 35 USC 119 from Provisional Application Ser. No. 60/498,211, filed Aug. 26, 2003, under 35 USC 111(b). The disclosure of that provisional application is incorporated herein by reference.

### BACKGROUND OF THE INVENTION

1. Field of the Invention

This invention relates to a method of treating buildings or structures contaminated with mold growth or toxic mold.

2. Introduction to the Invention

Mold contamination of homes and other buildings is not a new problem, but recent outbreaks have warranted aggressive action and indicate a strong need for improved methods to treat mold contamination. It is well-known that sufficiently moist or damp conditions can result in mold growth on the surface of almost any building material. Mold growth can become visually recognizable, and mold may grow to the extent so as to disfigure building material or structure surfaces. Entire homes or buildings, or significant portions of homes or buildings, have been seriously damaged or destroyed by the rapid spread of toxic mold contamination. Serious health risks can also be associated with mold contamination in homes and other buildings.

Numerous species of mold, all of which have the potential to cause adverse health effects, have been isolated from damp buildings. Probably the most well-known ailment associated with mold contamination is “sick building syndrome”. Molds can produce allergens that trigger allergic reactions or asthma attacks and some molds are known to produce potent toxins and/or irritants. Mold exposure can also result in opportunistic infections and people with weakened immune systems may be more vulnerable to health effects associated with mold. Potential health concerns are an important reason to decontaminate building surfaces of any indoor mold growth.

Development of indoor mold contamination can be influenced by many factors, of which moisture is the most important. Moisture problems in buildings contribute significantly to toxic mold contamination, particularly if the moisture problem remains undiscovered for extended periods of time. Some moisture problems in buildings have been linked to changes in building construction practices, some of which have resulted in buildings that are tightly sealed, but may lack adequate ventilation, potentially leading to moisture buildup. Common sources or causes of moisture problems include roof leaks, deferred maintenance, condensation associated with high humidity or cold spots in the building, localized flooding due to plumbing failures or heavy rains, slow leaks in plumbing fixtures, malfunction or poor design of humidification systems, and inadequate ventilation. Uncontrolled humidity can be a source of moisture leading to mold growth, particularly in hot, humid climates. Additionally, high amounts of moisture may be generated directly by occupants through activities such as bathing, cleaning, or cooking. While mold can occur at many locations in a building, sheet rock and ceilings are particularly susceptible because they contain organic materials that, when wet, provide a constant food source for mold. Building materials, such as drywall, may not allow moisture to escape easily. Mold gradually destroys surfaces so eliminating mold growth early on can reduce damage to building materials, but left untreated, mold can contaminate an entire house.

In accordance with current mold remediation procedures, most mold-contaminated building material areas have to be removed and replaced, which is an expensive and major undertaking that is accompanied by large costs for both the homeowner and insurance companies. Additionally, areas in which mold remediation is occurring have to be isolated and unoccupied for extended periods of time during the treatment process.

Conventional decontamination methods currently available produce a wide variety of undesirable byproducts, secondary wastes, and toxic effects. Additionally, existing methods are limited in their ability to fully decontaminate surfaces or areas to which they are applied and mold growth often reoccurs after treatment with existing methods. Mold contamination has also been treated using extreme thermal temperatures (i.e., 90° C.) for extended periods of time. Such treatment cannot be used to directly reduce mold contamination on surfaces of building structures, is costly, and can result in damage to the building or other components of the building (e.g., electrical work or plastic components). Mold growth on building structures has also been treated using dry air at a temperature of 50-60° C. in an attempt to reduce indoor moisture content to levels not suitable for mold growth. This method, however, is not effective in inactivating mold spores and has to be repeated continuously to be effective, and is quite costly as a result. In view of the shortcomings of existing mold decontamination methods, there is a strong need for effective mold decontamination methods which limit chemical run-off, conserve resources, and fully decontaminate surfaces.

It would be advantageous to provide new methods to address the problem of toxic mold contamination of homes and other buildings. The present invention is an improvement over currently available measures for controlling mold contamination in buildings. A strong need exists for alternative methods for treating mold contamination of building structures which are effective, non-toxic, environmentally friendly, and cost-effective. Such an alternative method which is effective, safe, environmentally friendly, and economical in treating mold contamination of building structures is provided. Additionally, a method which is able to introduce ozone into very small areas, such as cracks and crevices and other small openings such as inside of walls, duct work, and the like, is provided. This method is based on utilizing ozone dissolved in water dispersed as a uniform, stable, ultrafine, mist or fog comprised of very small water droplets into mold contaminated areas and onto mold contaminated building material surfaces without damage to building materials, with said mist or fog generated using commercially available means.

Therefore, there exists a need for an effective, economical, and safe method to treat mold contamination in homes and other buildings.

### BRIEF SUMMARY OF THE INVENTION

One objective of the present invention is to provide a method for mold decontamination of various solid building materials that is effective, economical and safe. The present invention includes a method to treat mold contamination on building material surfaces that greatly reduces the necessity of removing large portions of building materials and which is economical and safe. More specifically, the present invention provides a method for mold decontamination through the use of oxidative processes.

The method according to the present invention has been developed with the objective of treating mold contamination in homes or other buildings or structures. The present invention describes a new method for effective mold decontamination and remediation. The invention relates to a method of using ozone in the decontamination of toxic mold contaminated structures. More particularly, the present invention relates to a method of utilizing a nebulizer or other generator, e.g., a commercially available nebulizer for spraying very fine, small droplets of ozonated water in mold contaminated areas or on mold contaminated material surfaces in buildings to treat toxic mold growth. In preferred embodiments, the very fine, small droplets of ozonated water exist primarily as a uniform stable ultrafine mist or fog. The very small water droplets are able to penetrate areas which are typically difficult to access, such as cracks and crevices and other small openings such as inside of walls, duct work, and the like. The present invention provides a safe, economical, and effective method of utilizing the antimicrobial and antifungal properties of ozone dissolved in very small water droplets dispersed in a mist or fog to treat mold contamination of structural elements in homes and other buildings. The present method is highly effective in treatment of toxic mold contamination, simple to implement, inexpensive, has no substantially detrimental effect on the home or building or surrounding environment, and facilitates treatment of areas not treatable by existing conventional mold decontamination methods.

Accordingly, in a first aspect, present invention provides a method of treating mold contamination in a building, the method comprising inhibiting mold growth and deactivating mold associated with a building material surface by dispersing dissolved ozone in very small water droplets, preferably in the form of a uniform ultrafine stable mist or fog, onto contaminated surfaces to inhibit mold growth. The fog or mist is comprised of very small water droplets containing dissolved ozone and can generated, for example, by a commercially available nebulizer or generator. The ultrafine fog nature of the ozonated water treatment is extremely gentle and will essentially cause no damage to the surface material. Additionally, in a preferred embodiment, the ultrafine fog or mist nature of the ozonated water treatment is comprised of very small particles so as to penetrate cracks and crevices and other small openings such as inside of walls, duct work, and the like. The concentration of dissolved ozone in the ozonated water spray is effective in controlling and managing the growth and spread of mildew, mold and other harmful microorganism contamination of homes or other buildings.

In a second aspect, the present invention provides a method of treating mold contamination in a building, the method comprising inhibiting mold growth and deactivating mold associated with building materials by dispersing dissolved ozone in a very small water droplets, preferably in the form of a uniform ultrafine stable mist or fog, into an area of the building, the area of the building being bordered by building materials having mold contamination.

One objective of the present invention is to provide a method for treating toxic mold contamination in homes or other buildings.

Another objective of this invention is to utilize known antimicrobial and antifungal properties of ozone in a method for treating toxic mold contamination in homes or other buildings.

Another objective of the present invention is to utilize a commercially available nebulizer or generator to generate a uniform, ultrafine, stable mist or fog comprised of very small water droplets containing dissolved ozone as a method for treating toxic mold contamination in homes or other buildings.

Another objective of this invention is to provide a method for treating toxic mold contamination in homes or other buildings that is environmentally friendly and safe to plants and animals as compared to existing traditional chemical treatments.

Another objective of the present invention is to generate a uniform, ultrafine, stable mist or fog comprised of very small water droplets containing dissolved ozone, said water droplets being of small enough diameter so as to penetrate cracks and crevices and other small openings such as inside of walls, duct work, and the like.

Another objective of this invention is to provide a method for treating toxic mold contamination in homes or other buildings that requires no external chemicals other than water and ozone.

Another objective of this invention is to provide a method for treating toxic mold contamination in homes or other buildings that can be substantially less costly than existing methods.

Another objective of this invention is to provide a method for treating toxic mold contamination for which there are limited available treatment methods.

Another objective of this invention is to provide a method for treating toxic mold contamination that minimizes removal of building materials contaminated with toxic mold, which is time-consuming and expensive.

The present invention describes methods which efficiently and effectively eliminates mold growth when the method of this invention is practiced.

### BRIEF DESCRIPTION OF THE DRAWINGS

The invention is illustrated by the following drawing.

FIG. 1 is a flowchart showing a method of the present invention.

### DETAILED DESCRIPTION OF THE INVENTION

Removal of toxic mold contamination in buildings is a significant and growing problem. Completely effective approaches for mold remediation have not yet been developed. Despite currently available mold removal products and procedures, existing treatments are relatively ineffective in ridding a residence or other building of toxic mold. The method according to the present invention has been developed with the objective of effectively treating mold contamination of a residence or other building or structure.

In sufficient concentrations, ozone is known to destroy bacteria, fungi, viruses and other microorganisms. According to documents published by the Environmental Protection Agency, as little as 0.3 ppm ozone is sufficient to inhibit mold growth. Various methods and. processes using the known antimicrobial properties of ozone have been developed for disinfecting air and surfaces using ozone gas or ozonated water. Ozone has been used as an effective oxidizing agent in purifying drinking water (typically at ˜1 ppm), swimming pools, industrial waste, and as an oxidizing agent in chemical processes, among other uses. Ozonated water can be used for sanitizing surfaces without heat treatment and without the use of potentially harmful chemicals. Unlike other agents, treatment with ozonated water does not leave behind a residue that later needs to be removed from the building surface.

The current invention describes a use of ozone, more specifically dissolved ozone in water, for treating the common problem of toxic mold infestation in homes and other buildings. Specifically, the current invention describes a method for using ozonated water to treat toxic mold contamination in homes and other buildings using a nebulizer which disperses an ultrafine fog or mist comprised of very small water particles (5-20 um in diameter) containing dissolved ozone. These nebulizer devices are well-known, commercially available, and are described for example in U.S. Pat. Nos. 4,161,282, 3,993,246, 4,108,387, 4,261,511.

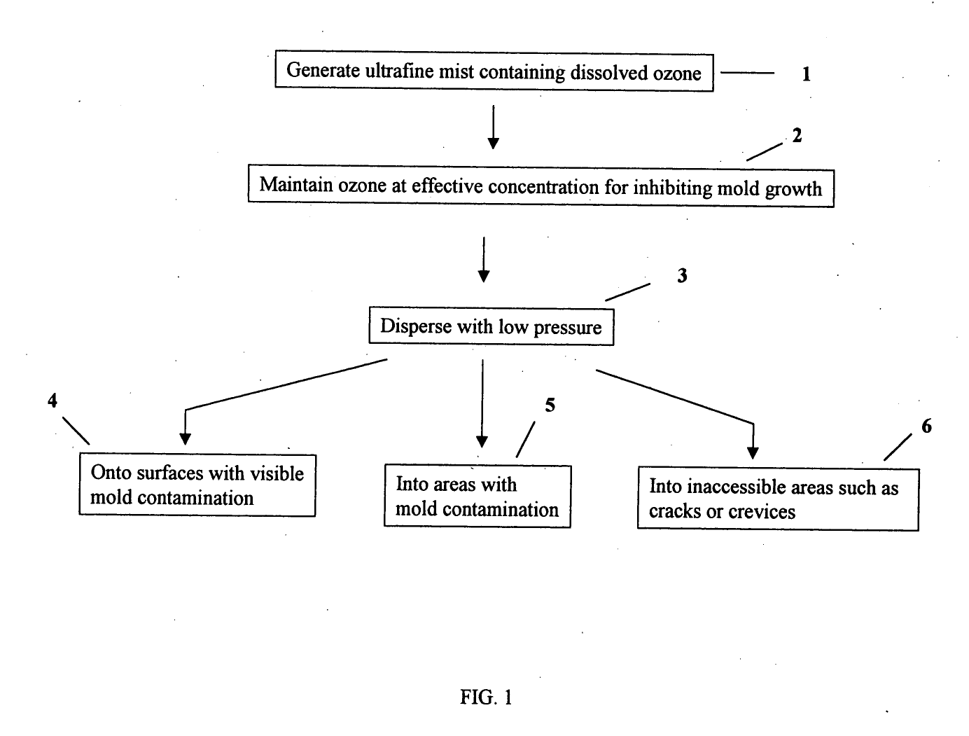

The present invention includes a method for effectively treating toxic mold contamination in homes or other buildings without resulting in adverse effects or damage to building structure, human health, animals, plants, or the environment, at low cost. The method is illustrated in FIG. 1, and comprises generating a uniform, ultrafine, stable, mist or fog comprised of water particles of very small size containing dissolved ozone (Step 1). The uniform, ultrafine, stable, mist or fog contains dissolved ozone at a concentration effective in inhibiting growth of mold on building surfaces (Step 2). The uniform, ultrafine, stable, mist or fog is dispersed under very low pressure so as not to cause damage to building surfaces (Step 3). The ultrafine mist or fog can be dispersed directly onto building surfaces containing visible mold contamination (Step 4). The ultrafine mist or fog can be dispersed into building areas containing mold contamination (Step 5). The ultrafine mist or fog can penetrate inaccessible areas, such as cracks or crevices (Step 6).

Ozone treatment of mold contamination in accordance with the present invention utilizes a commercially available nebulizer or generator to produce a uniform, ultrafine, stable mist or fog comprised of very small (e.g., 5-20 microns in diameter) ozonated water droplets which, substantially directly after being formed, is used to treat toxic mold contamination such as in homes and buildings and the like. The present invention uses very small water droplets containing dissolved ozone dispersed as a stable fog substantially directly into the area of mold contamination. The ozonated water droplets can be applied directly to a mold-contaminated surface, such as a building material surface, or to an area bordered by or surrounded by mold-contaminated structures. Unlike existing methods to treat mold contamination, the present method does not involve the use of harsh chemical treatments, heat, or removal of large portions of buildings. The present invention describes a method of treating mold contamination comprised of inactivating mold growth associated with a building material surface by dispersing a uniform, ultrafine, stable fog comprised of very small water droplets containing dissolved ozone onto the mold contaminated surface or into the area of mold contamination in an amount which is sufficient to inactivate said mold contamination but essentially without causing damage to building material. In addition, water droplets have a very small diameter and are light in weight so as to be able penetrate spaces that larger diameter and heavier water particles can not access, such as cracks and crevices and other small openings such as inside of walls, duct work, and the like.

The commercially available nebulizer or generator device used in the present invention generates a uniform, ultrafine, stable mist or fog of very small water particles, preferably having a diameter of between 5-20 microns and containing dissolved ozone, which results in a uniform dispersion of very small, ozonated water droplets having the appearance of a natural fog to disperse ozone uniformly in areas contaminated with mold to treat mold contamination, as well as in very small spaces, such as cracks and crevices and other small openings such as inside of walls, duct work, and the like. This method allows for wide dispersion of ozone for inhibiting mold growth. This method is equally effective in large areas, such as under roofs, as well as in small, confined spaces, such as crawl spaces. By using very small droplets, sufficient ozone can be delivered with adding only minimal amounts of water to the contaminated area. The low volume of water used allows for fast and efficient drying after application. The commercially available nebulizer or generator can be a hand-held device to disperse fog or mist, which is advantageous in homes and buildings in areas where alteration of application direction may be necessary. The nebulizer device utilized in the present invention can be readily moved, tilted, inverted, or vibrated during use without interfering with fog emission, which is advantageous for treating mold contamination in small spaces and which is ideal for toxic mold treatment methods described in the current invention. Also, the amount and density of fog can be readily controlled and varied to adapt to the area to which it is being applied. The ozone/water concentration in the dispersed fog can be varied without increasing pressure or volume of gas, which is advantageous in confined areas such as homes or other buildings.

Practically any surface of the building construction, whether it is a directly visible surface or a hidden, not immediately accessible, surface such a crawl space, may become contaminated under conditions favoring development of mold growth. The method according to the present invention is applicable to treatment of both directly accessible surfaces and hidden building surfaces. The method of the invention can be applied not only to surfaces delimiting rooms in a building, i.e. walls, ceilings, doors, windows and floors, but also to surface materials in crawl spaces and accessible parts of ventilation systems. Additionally, this method can be applied to very difficult to access areas, such as cracks and crevices and other small openings such as inside of walls, duct work, and the like.

In some situations, mold growth may have caused irreparable damage to building materials and structures such as gypsum wallboard, sheet rock and ceiling areas, carpet, windowsills, bathroom walls and ceilings. In such situations, it may be desirable to remove the damaged material and structural support component prior to implementation of the present invention. In order to achieve high rates of mold inhibition, it may be advantageous to subject the material surface to a pre-treatment step whereby mold growth present is at least partially removed from said building surface. Such a pretreatment step may be a conventional cleaning procedure using a liquid cleaning medium or it may be a vacuum cleaning step. Accordingly, after treatment with dissolved ozone in very small droplets, subsequent removal of mold spore-carrying droplets by some means, such as by vacuum cleaning, may be a further measure contributing to control of mold contamination.

The treatment method described in the present invention is likely to be very effective in controlling toxic mold contamination in homes and other buildings. In addition, the method of the present invention is environmentally friendly and does not harm humans, animals, or plants. The described method disperses dissolved ozone in very small water droplets onto building materials or into areas substantially uniformly and at concentrations effective to destroy mold growth. The described method does not cause damage to building materials.

The invention described herein provides an effective, very safe, and low-cost method for treatment of toxic mold contamination in homes or other buildings. This process does not damage structural elements and building materials, and harmful ozone gas is not released into the environment. The present invention provides a method to treat toxic mold contamination and the damaging effects mold contamination has on buildings and on human health in an efficient, economical, and environmentally friendly manner. Existing methods to treat toxic mold contamination utilize harsh chemicals or heat, require extensive removal of contaminated building materials, and do not completely eradicate mold contamination. Novel features of the present invention include a method which disperses ozone dissolved in very small water droplets in a uniform, ultrafine, stable, mist or fog at concentrations sufficient to effectively inhibit mold growth and penetrate very small spaces, such as cracks and crevices and other small openings such as inside of walls, duct work, and the like, but not damage building materials.

1. A method for treating mold contamination in a building comprising:
dispersing small water droplets containing dissolved ozone onto a material surface of the building, the surface comprising mold contamination;
the small water droplets having an ozone concentration sufficient to inactivate toxic mold contamination without causing substantial damage to the surface material.

2. The method according to claim 1, wherein said water droplets are about 5-20 micrometers in diameter.

3. The method of claim 1, wherein said water droplets containing dissolved ozone are generated using a nebulizer or generator.

4. The method of claim 1, wherein said water droplets containing dissolved ozone are dispersed in an ultrafine stable mist or fog using a nebulizer or generator.

5. The method of claim 4, wherein said ultrafine stable mist or fog is uniform in nature.

6. The method of claim 4, wherein said ultrafine, stable, mist or fog is dispersed under low pressure as a gentle mist or fog so as not to damage surface material.

7. The method of claim 4, wherein said uniform, ultrafine, stable, mist or fog comprised of small water droplets containing dissolved ozone is applied directly onto mold growth on the material surface of the building.

8. The method of claim 1, wherein the small droplets are of small size such that they can substantially penetrate crevices and other small openings, such as inside of walls, duct work, and the like.

9. The method of claim 1, wherein the material surface comprises a roof construction surface, a ceiling, a floor, a surface defining crawl space, a wall surface, or a surface which is not immediately accessible.

10. The method of claim 1, wherein mold growth is at least partially removed from said building surface prior to dispersing small water droplets containing dissolved ozone.

11. The method of claim 1, wherein a level of ozone utilized is sufficiently low so as to result in no substantial impact on environment, human health, animals, or plants.

12. A method for treating mold contamination in a building comprising:
dispersing small water droplets containing dissolved ozone into an area of the building, the area having surrounding building material contaminated with mold, and the small water droplets having an ozone concentration sufficient to inactivate toxic mold contamination without causing substantial damage to the building material.

13. The method of claim 12, wherein the area is substantially enclosed by surrounding building material.
